(19) United States
(12) Patent Application Publication
(10) Pub. No.: US 2010/0056917 A1
(43) Pub. Date: Mar. 4, 2010

(54) ULTRASONIC DIAGNOSTIC APPARATUS

(75) Inventor: Hiroyuki KARASAWA, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/547,236

(22) Filed: Aug. 25, 2009

(30) Foreign Application Priority Data
Aug. 26, 2008 (JP) 2008-216998

(51) Int. Cl. A61B 8/14

(52) U.S. Cl. 600/443

(57) ABSTRACT

An ultrasonic diagnostic apparatus in which an ultrasonic image of a puncture needle can be clearly captured. The apparatus includes: a reception signal processing unit for processing reception signals outputted from ultrasonic transducers; a reception beamformer for performing reception focusing processing on the reception signals to generate sound ray signals; an image signal generating unit for generating an image signal based on the sound ray signals; and a scan control unit for controlling a drive signal generating unit such that an ultrasonic beam with an intensity distribution having a center in a first direction is transmitted from a selected first group of ultrasonic transducers, and controls the reception beamformer such that an image signal representing an ultrasonic image in a second direction different from the first direction is generated based on reception signals outputted from a selected second group of ultrasonic transducers which receive ultrasonic echoes.

### CROSS-REFERENCE TO RELATED APPLICATION

The present application claims priority from Japanese Patent Application No. 2008-216998 filed on Aug. 26, 2008, the contents of which are incorporated herein by reference in their entirety.

### BACKGROUND OF THE INVENTION

1. Field of the Invention

The present invention relates to an ultrasonic diagnostic apparatus for imaging organs or bones within a living body and so on by transmitting and receiving ultrasonic waves to generate ultrasonic images to be used for diagnoses.

2. Description of a Related Art

In medical fields, various imaging technologies have been developed for observation and diagnoses within an object to be inspected. Especially, ultrasonic imaging for acquiring interior information of the object by transmitting and receiving ultrasonic waves enables image observation in real time and provides no exposure to radiation unlike other medical image technologies such as X-ray photography or RI (radio isotope) scintillation camera. Accordingly, ultrasonic imaging is utilized as an imaging technology at a high level of safety in a wide range of departments including not only the fetal diagnosis in obstetrics but also gynecology, circulatory system, digestive system, and so on.

The ultrasonic imaging is an image generation technology utilizing the nature of ultrasonic waves to be reflected at a boundary between regions with different acoustic impedances (e.g., a boundary between structures). Typically, an ultrasonic imaging apparatus (or also referred to as an ultrasonic diagnostic apparatus or ultrasonic observation apparatus) is provided with an ultrasonic probe to be used in contact with the object or an ultrasonic probe to be used by being inserted into a body cavity of the object. Further, in recent years, an ultrasonic endoscope in combination of an endoscope for optical observation within the object and an ultrasonic probe for intracavity has been also used.

In a typical ultrasonic probe, a vibrator (piezoelectric vibrator) having electrodes formed on both sides of a material having a piezoelectric property (a piezoelectric material) is used as an ultrasonic transducer for transmitting and receiving ultrasonic waves. When a pulsed or continuous wave voltage is applied to the vibrator, the piezoelectricmaterial expands and contracts to generate pulsed or continuous wave ultrasonic waves. Further, the vibrator expands and contracts by receiving propagating ultrasonic waves to generate an electric signal. This electric signal is used as a reception signal of the ultrasonic waves.

By using the ultrasonic probe, the object is scanned with an ultrasonic beam formed by synthesizing plural ultrasonic waves, and ultrasonic echoes reflected within the object are received. Further, reception focusing processing is performed, and thereby, image information on structures (e.g., internal organs, diseased tissues, and soon) existing within the object is obtained based on intensity of the ultrasonic echoes.

In ultrasonic imaging, when many structures existing within an organ serve as echo sources and a group of ultrasonic pulses are added to one another, an echo pattern (referred to as “speckle pattern” or the like) in which light points and dark points are dispersed due to interference of waves appears. Reducing the speckle pattern remains a problem to be solved.

As a related technology, Japanese Patent Application Publication JP-P2006-55282A discloses an ultrasonic diagnostic apparatus intended to appropriately and successfully reduce the influence of speckle noise generated when scattered waves generated by scatterers within the object cause phase interference with one another. The ultrasonic diagnostic apparatus includes ultrasonic transmitting means for transmitting ultrasonic waves, which are not plane waves and having no focused wavefront, to the object so that the wavefront temporally changes on the same scanning line, image signal generating means for receiving respective reflected waves with respect to each wavefront to generate respective image signals, and signal adding means for adding the signals obtained from the respective reflected waves generated by transmission ultrasonic waves having different wavefronts on the same scanning line so as to obtain an image signal with reduced speckle noise.

Japanese Patent Application Publication JP-P2006-340890A discloses an ultrasonic diagnostic apparatus for generating ultrasonic image data with reduced speckles or ultrasonic image data in which both the improvement in frame rate and the improvement in image quality are realized. The ultrasonic diagnostic apparatus includes generating means for controlling a piezoelectric element array to repeat ultrasonic transmission and reception performed at the same time with respect to plural different directions to generate plural ultrasonic image data in which scanning areas partially or wholly overlap, and speckle reducing means for synthesizing part or all of the generated ultrasonic image data with respect to part or all of overlapping areas to generate ultrasonic image data with reduced speckles.

Japanese Patent Application Publication JP-P2007-504876A (International Publication WO 2005/024462 A1) discloses an ultrasonic diagnostic imaging system for generating spatially compounded images. The ultrasonic diagnostic imaging system includes an array transducer, a multiline transmission beamformer coupled to the array transducer, for acting to cause the array transducer to transmit multiple beams in different directions during a signal transmitting/receiving interval, a multiline reception beamformer coupled to the array transducer, for acting to form echo signals of reception beams in the different directions according to echoes received during the single transmitting/receiving interval, a combiner for acting to combine signals relating to a common point in image fields received from the different directions according to the echo signals, and a display coupled to the combiner, for displaying a spatially compounded image.

According to the spatial compounding method of obtaining an ultrasonic image by combining signals relating to a common point in image fields received from different directions, speckle patterns can be reduced, but there are problems that the frame rate becomes lower, motion artifacts are generated, and so on. In order to solve the problems, JP-P2006-55282A, JP-P2006-340890A, and JP-P2007-504876A propose that the frame rate is prevented from being lower by transmitting ultrasonic waves having random wavefront, or simultaneously transmitting ultrasonic beams in plural directions. However, if ultrasonic waves having random wavefronts are transmitted, the basic image quality is deteriorated. Further, if ultrasonic beams are simultaneously transmitted in plural directions, the influence of crosstalk is unavoidable.

Moreover, in the ultrasonic diagnostic apparatus, including the case of using the ultrasonic endoscope, it is necessary to clearly capture an ultrasonic image of a puncture needle. However, ultrasonic waves are diffusely reflected by the puncture needle, and it is difficult to clearly capture the ultrasonic image of the puncture needle in typical ultrasonic imaging.

Japanese Patent Application Publication JP-P2004-208859A discloses an ultrasonic diagnostic apparatus in which visualized body tissues and a puncture needle image can be easily distinguished by locating a puncture needle within a living body and processing of highlighting an ultrasonic image thereof. The ultrasonic diagnostic apparatus includes an ultrasonic probe provided with a puncture adapter having angle detecting means for defining an insertion path of a puncture needle on a scanning line of an ultrasonic beam and detecting an insertion angle of the puncture needle relative to a living body, and provided with plural vibrators for generating ultrasonic waves, the ultrasonic probe scanning within the living body by using the ultrasonic waves generated from the vibrators as an ultrasonic beam, and receiving reflected waves thereof, ultrasonic image generating means for generating an ultrasonic image within the living body based on the reflected waves of the ultrasonic beam received by the ultrasonic probe, display means for displaying the ultrasonic image generated by the ultrasonic image generating means, ultrasonic beam deflecting means for deflecting the ultrasonic beam in a direction substantially orthogonal to the puncture path of the puncture needle based on the puncture angle of the puncture needle relative to the living body detected by the angle detecting means, and ultrasonic beam control means for controlling the ultrasonic beam deflecting means to deflect the ultrasonic beam in the direction substantially orthogonal to the puncture path of the puncture needle at intervals of predetermined times of scanning.

According to JP-P2004-208859A, the ultrasonic image of the puncture needle can be highlighted in display, but the configuration and operation of the ultrasonic diagnostic apparatus becomes complicated because it is necessary to perform special transmission so that the ultrasonic beam is deflected in the direction substantially orthogonal to the puncture path of the puncture needle.

### SUMMARY OF THE INVENTION

The present invention has been achieved in view of the above-mentioned points. A purpose of the present invention is to provide an ultrasonic diagnostic apparatus in which an ultrasonic image of a puncture needle can be clearly captured or speckle patterns can be reduced without complication of configuration and operation of the ultrasonic diagnostic apparatus.

In order to accomplish the above-mentioned purpose, an ultrasonic diagnostic apparatus according to one aspect of the present invention includes: an ultrasonic probe including plural ultrasonic transducers for transmitting ultrasonic waves toward an object to be inspected according to drive signals and receiving ultrasonic echoes propagating from the object to output reception signals; a drive signal generating unit for supplying the drive signals to the plural ultrasonic transducers, respectively; a reception signal processing unit for processing the reception signals respectively outputted from the plural ultrasonic transducers; a reception beamformer for performing reception focusing processing on the reception signals outputted from the reception signal processing unit to generate sound ray signals; image signal generating means for generating an image signal based on the sound ray signals generated by the reception beamformer; and scan control means for controlling the drive signal generating unit such that an ultrasonic beam with an intensity distribution having a center in a first direction is transmitted from a selected first group of ultrasonic transducers, and controlling the reception beamformer such that an image signal representing an ultrasonic image in a second direction different from the first direction is generated based on reception signals outputted from the reception signal processing unit and derived from a selected second group of ultrasonic transducers which receive ultrasonic echoes.

According to the one aspect of the present invention, an ultrasonic image of a puncture needle can be clearly captured by transmitting an ultrasonic beam with an intensity distribution having a center in a first direction from the selected first group of ultrasonic transducers, and generating an image signal representing an ultrasonic image in a second direction different from the first direction based on reception signals outputted from the reception signal processing unit and derived from the selected second group of ultrasonic transducers which receive ultrasonic echoes. Further, speckle patterns can be reduced in the case of synthesizing ultrasonic images based on plural sets of reception signals with respect to a common point, which signals are outputted from the reception signal processing unit and derived from ultrasonic transducers in plural reception apertures receiving ultrasonic echoes.

### BRIEF DESCRIPTION OF THE DRAWINGS

FIG. 1 is a block diagram showing a configuration of an ultrasonic diagnostic apparatus according to the first embodiment of the present invention;

FIG. 2 shows a relationship between the direction of an ultrasonic beam transmitted from ultrasonic transducers and the ultrasonic image formation direction;

FIG. 3A is a plan view showing a top surface of a leading end of an insertion part of an ultrasonic endoscope;

FIG. 3B is a side sectional view showing a side surface of the leading end of the insertion part of the ultrasonic endoscope;

FIG. 4 shows an ultrasonic beam transmitted from ultrasonic transducers and reception curves representing timings when ultrasonic echoes are received by the ultrasonic transducers;

FIG. 5 shows ultrasonic beams transmitted at plural times from ultrasonic transducers and reception curves representing timings when ultrasonic echoes are received by the ultrasonic transducers;

FIG. 6 shows the case where an image signal at all depths can not be obtained by one transmission; and

FIG. 7 shows an ultrasonic beam transmitted from ultrasonic transducers and reception curves representing timings when ultrasonic echoes are received by the ultrasonic transducers.

### DESCRIPTION OF THE PREFERRED EMBODIMENTS

Hereinafter, embodiments of the present invention will be explained in detail with reference to the drawings. The same reference numerals are assigned to the same component elements and the explanation thereof will be omitted.

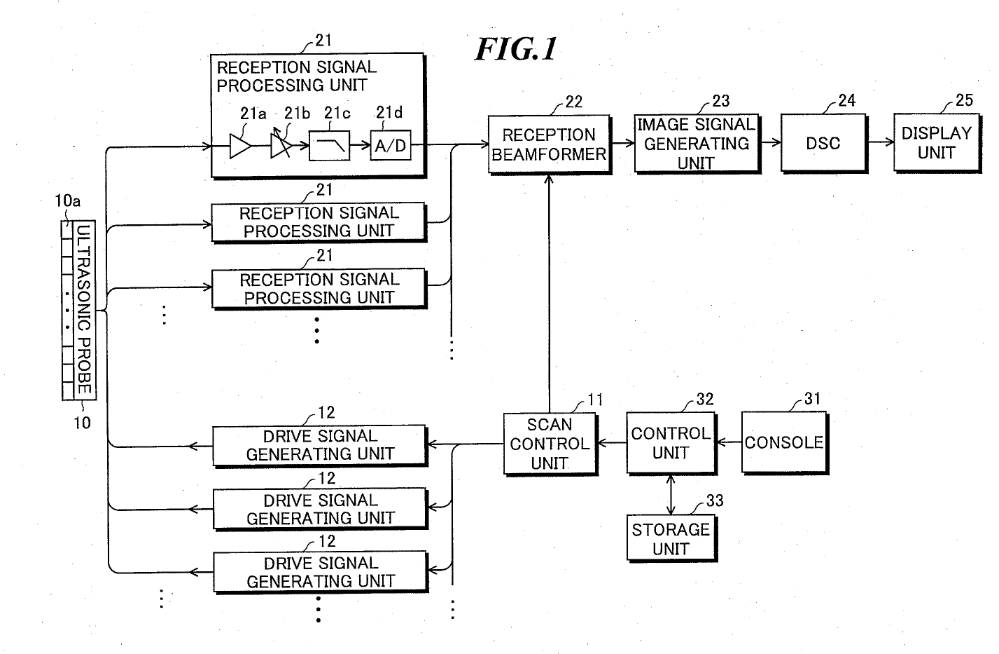

FIG. 1 is a block diagram showing a configuration of an ultrasonic diagnostic apparatus according to the first embodiment of the present invention. The ultrasonic diagnostic apparatus includes an ultrasonic probe 10, a scan control unit 11, drive signal generating units 12, reception signal processing units 21, a reception beamformer 22, an image signal generating unit 23, a DSC 24, a display unit 25, a console 31, a control unit 32, and a storage unit 33.

The ultrasonic probe 10 includes plural ultrasonic transducers (hereinafter, also simply referred to as “elements”) 10a that transmit ultrasonic waves to an object to be inspected according to applied drive signals, and receive ultrasonic echoes propagating from the object to output reception signals. These ultrasonic transducers 10a are one-dimensionally or two-dimensionally arranged to form a transducer array.

Each ultrasonic transducer includes a vibrator having electrodes formed on both ends of a material having a piezoelectric property (piezoelectric material) such as a piezoelectric ceramic represented by PZT (Pb (lead) zirconate titanate), a polymeric piezoelectric element represented by PVDF (polyvinylidene difluoride). When a pulsed or continuous wave voltage is applied to the electrodes of the vibrator, the piezoelectric material expands and contracts. By the expansion and contraction, pulse or continuous wave ultrasonic waves are generated from the respective vibrators, and an ultrasonic beam is formed by synthesizing these ultrasonic waves. Further, the respective vibrators expand and contract by receiving the propagating ultrasonic waves to generate electric signals. These electric signals are outputted as reception signals of ultrasonic waves.

The scan control unit 11 can set a transmission direction, a reception direction, a focal depth, and an aperture diameter of the ultrasonic transducer array when an imaging region within the object is scanned with the ultrasonic beam. The scan control unit 11 controls the drive signal generating units 12 and the reception beamformer 22 according to the settings.

When ultrasonic waves are transmitted, the scan control unit 11 sets delay times (a delay pattern) to be provided to the drive signals in order to perform transmission focusing processing according to the transmission direction and the focal depth of the ultrasonic beam to be transmitted from the ultrasonic probe 10 and the aperture diameter.

The drive signal generating unit 12 has plural channels. Each channel includes a pulser or the like for generating a drive signal to be supplied to the selected ultrasonic transducer according to the delay time set in the scan control unit 11.

The reception signal processing unit 21 has plural channels. Each channel includes a preamplifier 21a, a variable gain amplifier 21b, a low-pass filter 21c, and an A/D converter 21d. The reception signal outputted from the ultrasonic transducer is amplified by the preamplifier 21a and the variable gain amplifier 21b, band-limited by the low-pass filter 21c, and converted into digital reception signal (RF data) by the A/D converter 21d.

The reception beamformer 22 has plural delay patterns (phase matching patterns) according to reception directions and focal depths of ultrasonic echoes, and performs reception focusing processing by providing respective delays to the reception signals outputted from the reception signal processing units 21 according to the reception direction and focal depth set by the scan control unit 11 and adding those reception signals to one another. By the reception focusing processing, sound ray signals in which the focus of the ultrasonic echoes is narrowed are formed with respect to the reception directions.

The image signal generating unit 23 performs envelope detection processing on the sound ray signals and further performs preprocess processing such as Log (logarithm) compression and gain adjustment to generate a B-mode image signal. The DSC 24 converts (raster-converts) the generated B-mode image signal into an image signal for display that follows the normal scan system of television signals. Thereby, an ultrasonic image is displayed on the display unit 25.

The console 31 includes a keyboard, an adjustment knob, a mouse, and so on, and is used by an operator for inputting commands and information to the ultrasonic diagnostic apparatus. The control unit 32 controls the respective units of the ultrasonic diagnostic apparatus according to the commands and information inputted by using the console 31. In the embodiment, the scan control unit 11, the reception beamformer 22 to the DSC 24, and the control unit 32 are formed of a central processing unit (CPU) and software for allowing the CPU to execute various kinds of processing, however, they may be formed of digital circuits or analog circuits. The software is stored in the storage unit 33. As a recording medium in the storage unit 33, not only a built-in hard disk but also a flexible disk, MO, MT, RAM, CD-ROM, DVD-ROM, or the like may be used.

In the embodiment, the scan control unit 11 controls the drive signal generating units 12 such that an ultrasonic beam with an intensity distribution having a center in a first direction is transmitted from a selected first group of ultrasonic transducers, and controls the reception beamformer 22 such that an image signal representing an ultrasonic image in a second direction different from the first direction is generated based on the reception signals outputted from the reception signal processing units 21 and derived from a selected second group of ultrasonic transducers which receive ultrasonic echoes.

Here, it is desirable that the scan control unit 11 controls the reception beamformer 22 such that an image signal representing an ultrasonic image in a space region, in which energy of the ultrasonic beam transmitted from the selected ultrasonic transducers not less than one tenth of the energy in the first direction exists, is generated. Thereby, an ultrasonic image with a good S/N ratio can be obtained.

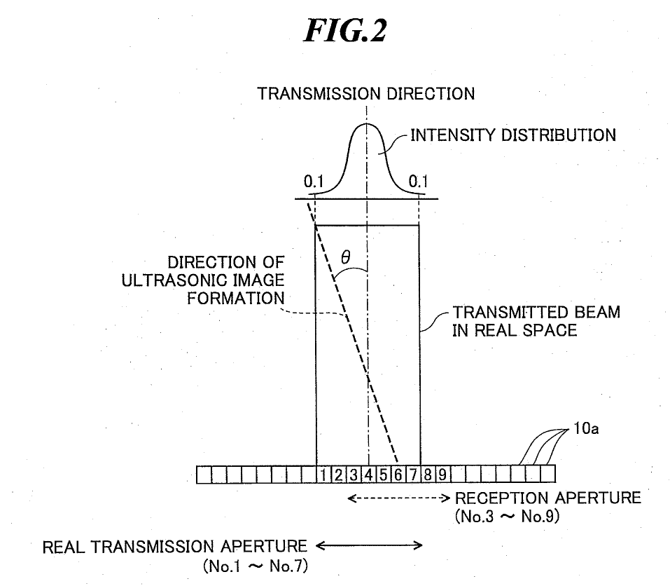

FIG. 2 shows a relationship between the direction of an ultrasonic beam transmitted from ultrasonic transducers and the direction of ultrasonic image formation. The first group of ultrasonic transducers (elements No. 1 to No. 7) in a real transmission aperture are selected from among the plural ultrasonic transducers 10a included in the ultrasonic probe, and an ultrasonic beam in a real space is transmitted from the first group of ultrasonic transducers into the first direction (the direction substantially orthogonal to the surface on which the ultrasonic transducers 10a are arranged in FIG. 2). This ultrasonic beam has a broader width than that of a typical ultrasonic beam, and a broad intensity distribution having a center in the first direction is formed.

Generally, an ultrasonic image in the first direction is formed when an ultrasonic beam is transmitted in the first direction. On the other hand, in the embodiment, as shown in FIG. 2, an ultrasonic image in the second direction different from the first direction is formed based on reception signals obtained by the second group of ultrasonic transducers (elements No. 3 to No. 9) in a reception aperture receiving ultrasonic echoes.

In FIG. 2, an angle formed by the second direction and a direction of a perpendicular line of the surface, on which the ultrasonic transducers 10a are arranged, is shown as θ. Assuming that N ultrasonic transducers are one-dimensionally arranged in the reception aperture, the waveform v(i) of the reception signal outputted from the i-th ultrasonic transducer from the left in the reception aperture is expressed by the following equation (1) by using function “f” of time “t” with a parameter of angular frequency “ω”.

v(i)=α(i)·f{ωt+φ(i)} (1)

Here, amplitude α(i) and phase φ(i) vary depending on ultrasonic transducers.

When reception focusing processing is simply performed on the reception signals expressed by the equation (1), an ultrasonic image in the direction of the perpendicular line of the surface, on which the ultrasonic transducers 10a are arranged, can be obtained. As shown in FIG. 2, in order to tilt the direction of the ultrasonic image counterclockwise by the angle θ, the phase of the reception signal may be shifted as expressed by the following equation (2) by using phase difference Δφ corresponding to the angle θ.

v′(i)=α(i)·f{ωtφ(i)+(N−i)Δφ} (2)

Thereby, in an ultrasonic endoscope, for example, it becomes easy to clearly capture an ultrasonic image of a puncture needle.

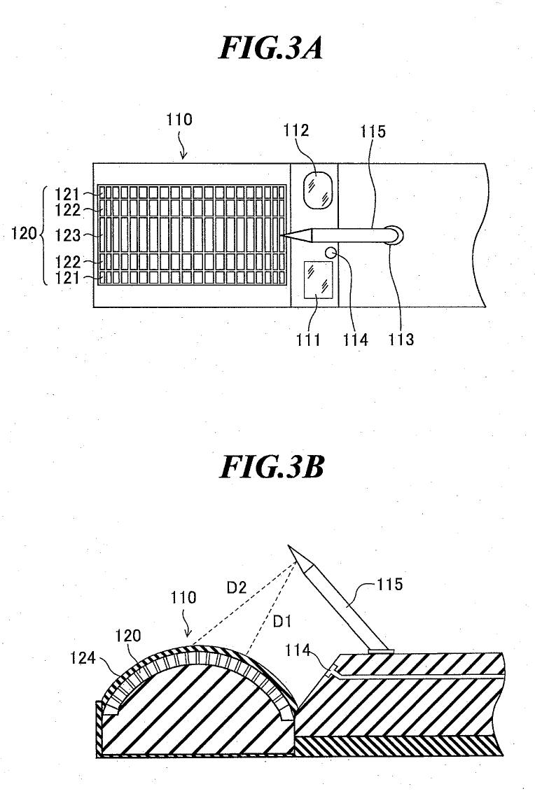

FIG. 3A is a plan view showing a top surface of a leading end of an insertion part of an ultrasonic endoscope, and FIG. 3B is a side sectional view showing a side surface of the leading end of the insertion part of the ultrasonic endoscope. In FIG. 3A, an acoustic matching layer 124 as shown in FIG. 3B is omitted.

As shown in FIGS. 3A and 3B, at the leading end of the insertion part of the ultrasonic endoscope, an ultrasonic transducer part 110 (corresponding to the ultrasonic probe 10 in FIG. 1), an observation window 111, an illumination window 112, a treatment tool passage opening 113, and a nozzle hole 114 are provided. A punctuation needle 115 is provided in the treatment tool passage opening 113. In FIG. 3A, an objective lens is fit in the observation window 111, and an input end of an image guide or a solid-state image sensor such as a CCD camera is provided in the imaging position of the objective lens. These configure observation optics. Further, an illumination lens for outputting illumination light supplied from the light source unit via a light guide is fit in the illumination window 112. These configure illumination optics.

The treatment tool passage opening 113 is a hole for leading out a treatment tool or the like inserted from the treatment tool insertion opening provided in the operation part of the ultrasonic endoscope. Various treatments are performed within a body cavity of the object by projecting the treatment tool such as the punctuation needle 115 or forceps from the hole and operating it with the operation part. The nozzle hole 114 is provided for injecting a liquid (water or the like) for cleaning the observation window 111 and the illumination window 112.

The ultrasonic transducer part 110 includes a convex-type ultrasonic transducer array 120, and the ultrasonic transducer array 120 has plural ultrasonic transducers 121-123 arranged in five rows on a curved surface. An ultrasonic image of the punctuation needle 115 in the direction D1 is generally formed by the plural ultrasonic transducers transmitting and receiving ultrasonic waves along the direction D1. However, ultrasonic waves are not diffusely reflected by the punctuation needle 115, and it is difficult to clearly capture the ultrasonic image of the puncture needle 115 in that ultrasonic imaging.

Accordingly, in the embodiment, the ultrasonic image of the puncture needle 115 in the direction D2 different from the transmission direction of ultrasonic waves (e.g., the direction D1) is formed. Thereby, the ultrasonic image clearly capturing the leading end of the punctuation needle 115 can be formed. Alternatively, both the ultrasonic image of the object in the transmission direction of ultrasonic waves and the ultrasonic image of the punctuation needle in the direction different from the transmission direction of ultrasonic waves may be formed. In this case, the reception beamformer 22 as shown in FIG. 1 generates plural kinds of sound ray signals by performing plural kinds of reception focusing processing on the reception signals outputted from the reception signal processing units 21, and the image signal generating unit 23 generates an image signal representing the ultrasonic image of the object in the transmission direction of ultrasonic waves and an image signal representing the ultrasonic image of the punctuation needle in the direction different from the transmission direction of ultrasonic waves based on the plural kinds of sound ray signals generated by the reception beamformer 22. In the display unit 25 displays those plural ultrasonic images.

Next, the second embodiment of the present invention will be explained. In the second embodiment, reception focusing processing differs from that in the first embodiment, and the rest of the operation is the same as that in the first embodiment.

In the second embodiment, the scan control unit 11 as shown in FIG. 1 controls the reception beamformer 22 to generate a sound ray signal representing a sound ray passing though a point by phase-matching and adding reception signals outputted from the reception signal processing unit 21 and derived from the second group of ultrasonic transducers which receive ultrasonic echoes generated due to ultrasonic waves transmitted from the first group of ultrasonic transducers, based on a relationship between directions of the ultrasonic waves transmitted from the first group of ultrasonic transducers to the point and reception directions of the second group of ultrasonic transducers which receive ultrasonic echoes generated from the point due to the ultrasonic waves.

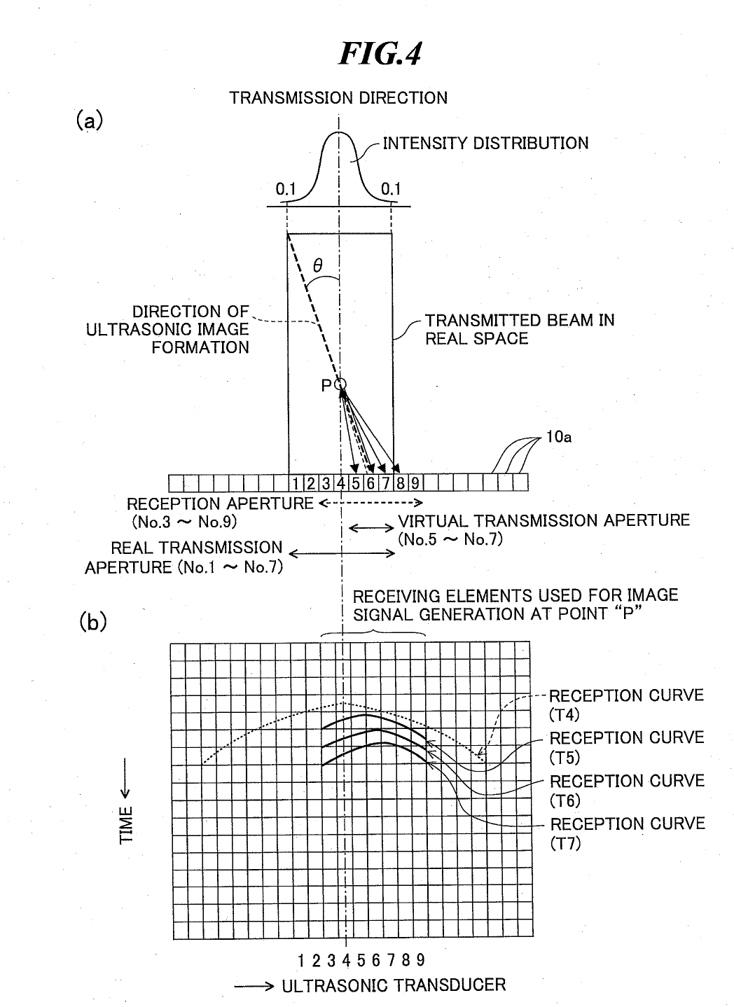

FIG. 4 shows an ultrasonic beam transmitted from ultrasonic transducers and reception curves representing timings when ultrasonic echoes are received by the ultrasonic transducers. FIG. 4(a) shows a relationship between the direction of the ultrasonic beam transmitted from the ultrasonic transducers and the direction of ultrasonic image formation. FIG. 4(b) shows the reception curves representing timings when ultrasonic echoes generated by reflection of transmitted ultrasonic waves at point P are received by the ultrasonic transducers.

As shown in FIG. 4(a), the first group of ultrasonic transducers (elements No. 1 to No. 7) in a real transmission aperture are selected from among the plural ultrasonic transducers 10a included in the ultrasonic probe, and an ultrasonic beam in a real space is transmitted from the first group of ultrasonic transducers into the first direction. This ultrasonic beam has a broader width than that of a typical ultrasonic beam, and a broad intensity distribution having a center in the first direction is formed. The transmitted ultrasonic waves are reflected at point P within the object and ultrasonic echoes are received by the second group of ultrasonic transducers (elements No. 3 to No. 9) in the reception aperture.

Here, distances from the respective ultrasonic transducers to the point P are different from one another and the transmitted ultrasonic beam is a broad beam in which the focal point is not narrowed on the point P, and thus, times when the ultrasonic waves transmitted from the respective ultrasonic transducers reach the point P are different from one another. As a result, as shown in FIG. 4(b), the reception curves in the same number as the number of ultrasonic transducers used for transmission are obtained. FIG. 4(b) shows the reception curve (T4) for the transmission waves of the element No. 4, the reception curve (T5) for the transmission waves of the element No. 5, the reception curve (T6) for the transmission waves of the element No. 6, and the reception curve (T7) for the transmission waves of the element No. 7.

The scan control unit 11 as shown in FIG. 1 sets a virtual transmission aperture in a part of the real transmission aperture based on the relationship between the directions of ultrasonic waves transmitted from the respective ultrasonic transducers in the real transmission aperture to the point P and the reception direction of the predetermined ultrasonic transducers in the reception aperture for receiving the ultrasonic echoes generated from the point P due to the ultrasonic waves. For example, the virtual transmission aperture is set such that the angle viewing the reception aperture from the point P where the ultrasonic waves are reflected and the angle viewing the virtual transmission aperture from the point P have a predetermined relationship (proportional relationship or the like). Note that the virtual transmission aperture is within the virtual transmission aperture. Further, it is desirable that the center of the virtual transmission aperture and the center of the reception aperture are aligned with the center of the direction of ultrasonic image formation.

As shown FIG. 4(b), the reception curves in the same number as the number of plural ultrasonic transducers (elements No. 5 to No. 7) in the virtual transmission aperture are obtained. Further, the scan control unit 11 controls the reception beamformer 22 to generate a sound ray signal representing a sound ray passing though the point P by phase-matching and adding reception signals outputted the reception signal processing units 21 from plural ultrasonic transducers (elements No. 3 to No. 9) in the reception aperture which receive ultrasonic echoes generated due to ultrasonic waves transmitted from the plural ultrasonic transducers in the virtual transmission aperture, according to the above-mentioned reception curves.

The reception beamformer 22 performs reception focusing processing by providing respective delays to the reception signals corresponding to the second group of ultrasonic transducers and adding those reception signals to one another, so as to generate sound ray signals. The image signal generating unit 23 generates a B-mode image signal based on the sound ray signals generated by the reception beamformer 22. As a result, the reception signals corresponding to the ultrasonic transmission signals of the transmitting elements aligned in the reception direction (direction of ultrasonic image formation) are extracted to generate an image signal, and thus, the image signal representing an ultrasonic image in the virtual transmission and reception direction different from the real transmission direction can be effectively extracted. Further, it is desirable to perform encoded transmission for discrimination of transmission waves from the respective elements.

Next, the third embodiment of the present invention will be explained. The third embodiment is different from the second embodiment in that transmission is performed at plural times for obtaining an ultrasonic image in one direction, and the rest of the operation is the same as that in the second embodiment.

In the third embodiment, the scan control unit 11 controls the drive signal generating units 12 such that the ultrasonic beams with an intensity distribution having a center in a first direction are transmitted at plural times from the ultrasonic transducers in sequentially selected plural transmission apertures, and controls the reception beamformer 22 such that an image signal representing an ultrasonic image in a second direction different from the first direction is generated based on the plural sets of reception signals outputted from the reception signal processing units 21 and derived from the ultrasonic transducers in the reception aperture which receive ultrasonic echoes generated due to the ultrasonic beams transmitted at plural times.

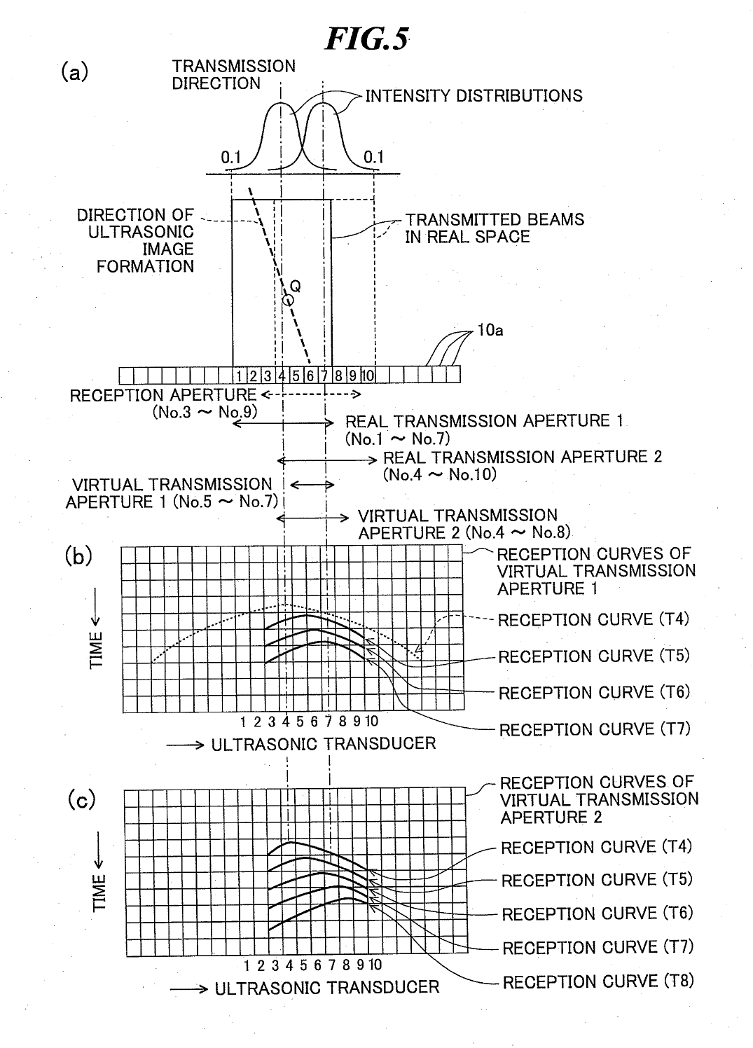

FIG. 5 shows ultrasonic beams transmitted at plural times from ultrasonic transducers and reception curves representing timings when ultrasonic echoes are received by the ultrasonic transducers. FIG. 5(a) shows a relationship between the directions of the ultrasonic beams transmitted at plural times from the ultrasonic transducers and the direction of ultrasonic image formation. FIG. 5(b) shows the reception curves representing timings when ultrasonic echoes generated by reflection of transmitted ultrasonic waves at point Q in the first transmission and reception are received by the ultrasonic transducers. FIG. 5(c) shows the reception curves representing timings when ultrasonic echoes generated by reflection of transmitted ultrasonic waves at point Q in the second transmission and reception are received by the ultrasonic transducers.

As shown in FIG. 5(a), first, ultrasonic transducers (elements No. 1 to No. 7) in a real transmission aperture 1 are selected from among the plural ultrasonic transducers 10a included in the ultrasonic probe, and an ultrasonic beam in a real space is transmitted from the ultrasonic transducers in the real transmission aperture 1 in a predetermined direction. The transmitted ultrasonic waves are reflected at point Q within the object and ultrasonic echoes are received by the plural ultrasonic transducers (elements No. 3 to No. 9) in the reception aperture.

The scan control unit 11 as shown in FIG. 1 sets a virtual transmission aperture 1 in a part of the real transmission aperture 1 based on the relationship between the directions of ultrasonic waves transmitted from the respective ultrasonic transducers in the real transmission aperture 1 to the point Q and the reception direction of the predetermined ultrasonic transducers in the reception aperture for receiving the ultrasonic echoes generated from the point Q due to the ultrasonic waves.

As shown FIG. 5(b), the reception curves in the same number as the number of plural ultrasonic transducers (elements No. 5 to No. 7) in the virtual transmission aperture 1 are obtained. FIG. 5(b) shows the reception curve (T4) for the transmission waves of the element No. 4, the reception curve (T5) for the transmission waves of the element No. 5, the reception curve (T6) for the transmission waves of the element No. 6, and the reception curve (T7) for the transmission waves of the element No. 7.

Further, the scan control unit 11 as shown in FIG. 1 controls the reception beamformer 22 to generate a sound ray signal representing a sound ray passing through the point Q by phase-matching and adding reception signals outputted from the reception signal processing units 21 and derived from plural ultrasonic transducers (elements No. 3 to No. 9) in the reception aperture which receive ultrasonic echoes generated due to ultrasonic waves transmitted from the plural ultrasonic transducers in the virtual transmission aperture 1, according to the above-mentioned reception curves.

Referring to FIG. 5(a) again, subsequently, ultrasonic transducers (elements No. 4 to No. 10) in a real transmission aperture 2 are selected from among the plural ultrasonic transducers 10a included in the ultrasonic probe, and an ultrasonic beam in a real space is transmitted from the ultrasonic transducers in the real transmission aperture 2 in a predetermined direction. The transmitted ultrasonic waves are reflected at point Q within the object and ultrasonic echoes are received by the plural ultrasonic transducers (elements No. 3 to No. 9) in the reception aperture.

The scan control unit 11 as shown in FIG. 1 sets a virtual transmission aperture 2 in a part of the real transmission aperture 2 based on the relationship between the directions of ultrasonic waves transmitted from the respective ultrasonic transducers in the real transmission aperture 2 to the point Q and the reception direction of the predetermined ultrasonic transducers in the reception aperture for receiving the ultrasonic echoes generated from the point Q due to the ultrasonic waves.

As shown FIG. 5(c), the reception curves in the same number as the number of plural ultrasonic transducers (elements No. 4 to No. 8) in the virtual transmission aperture 2 are obtained. FIG. 5(c) shows the reception curve (T4) for the transmission waves of the element No. 4, the reception curve (T5) for the transmission waves of the element No. 5, the reception curve (T6) for the transmission waves of the element No. 6, the reception curve (T7) for the transmission waves of the element No. 7, and the reception curve (T8) for the transmission waves of the element No. 8.

The scan control unit 11 as shown in FIG. 1 controls the reception beamformer 22 to generate a sound ray signal representing a sound ray passing through the point Q by phase-matching and adding reception signals outputted from the reception signal processing units 21 and derived from plural ultrasonic transducers (elements No. 3 to No. 9) in the reception aperture which receive ultrasonic echoes generated due to ultrasonic waves transmitted from the plural ultrasonic transducers in the virtual transmission aperture 2, according to the above-mentioned reception curves.

The reception beamformer 22 synthesizes the reception signals represented by the reception curves for the virtual transmission aperture 1 and the reception signals represented by the reception curves for the virtual transmission aperture 2, performs reception focusing processing on those reception signals to generate sound ray signals. The image signal generating unit 23 generates a B-mode image signal based on the sound ray signals generated by the reception beamformer 22. As a result, image signals can be generated by synthesizing the reception signals obtained by two transmissions, and the S/N ratio of the image signal is improved. Further, the virtual transmission aperture or the reception aperture can be made broader and resolving power can be improved.

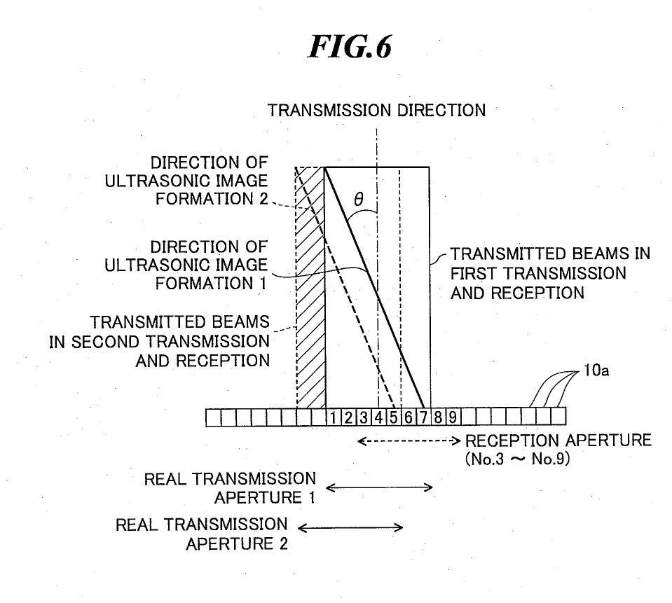

Furthermore, as shown in FIG. 6, it may be impossible to obtain an image signal of the entire imaging region by one transmission and reception depending on the position or direction of ultrasonic image formation. In this case, an image signal of the entire imaging region can be obtained by transmission and reception at plural times. As shown in FIG. 6, in the first transmission and reception, the real transmission aperture 1 is set, and an image in a region within a range of transmission beam as shown by a solid line can be obtained. The reception beamformer 22 performs reception focusing processing on the reception signals obtained in the first transmission and reception to generate sound ray signals. Then, in the second transmission and reception, the real transmission aperture 2 is set, and an image in a shaded region within a range of transmission beam as shown by a broken line can be obtained. The reception beamformer 22 synthesizes the reception signals obtained in the first transmission and reception and the reception signals obtained in the second transmission and reception, and performs reception focusing processing on those reception signals to generate sound ray signals.

In the above embodiments, at the respective times of ultrasonic beam transmission, reception processing of ultrasonic waves may be performed with respect to plural different virtual transmission apertures and reception apertures and an ultrasonic image may be formed based on the resulting plural sets of reception signals. In this case, an ultrasonic image is obtained by synthesizing signals with respect to a common point in image fields received from different directions, and the effect of reducing speckle patterns can be obtained similarly in the case of the spatial compounding method explained above.

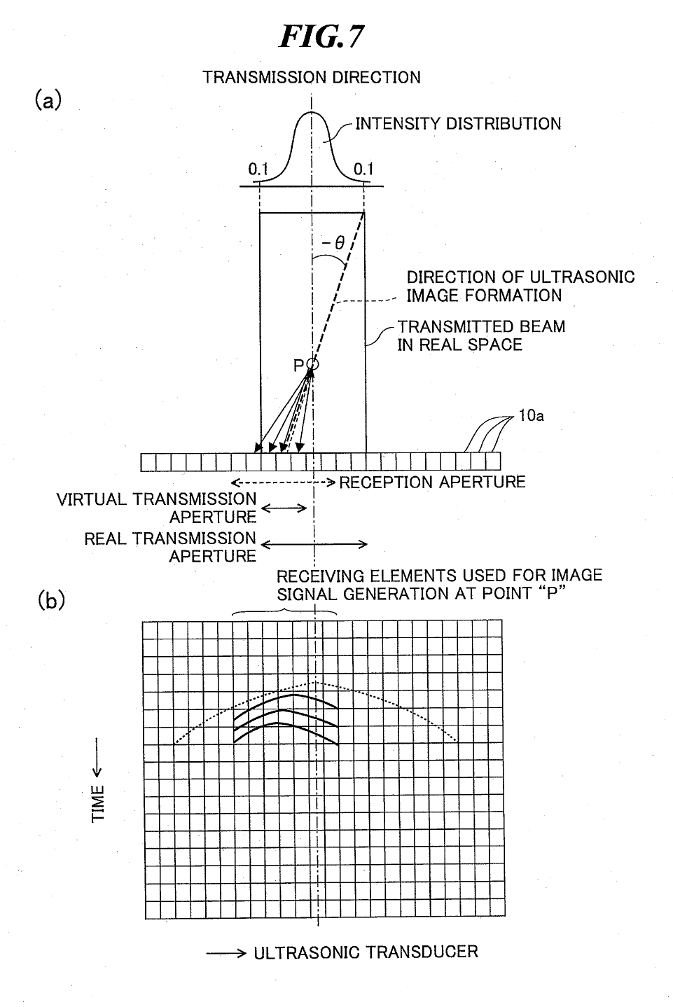

For example, after the ultrasonic beam is transmitted at once, two sets of reception signals are obtained by performing the first reception processing as shown in FIG. 4 and performing the second reception processing as shown in FIG. 7. FIG. 7(a) shows a relationship between the direction of the ultrasonic beam transmitted from the ultrasonic transducers and the direction of ultrasonic image formation. FIG. 7(b) shows the reception curves representing timings when ultrasonic echoes generated by reflection of transmitted ultrasonic waves at point P are received by the ultrasonic transducers. In this example, an angle formed by the direction of ultrasonic image formation in the first reception processing as shown in FIG. 4(a) is θ, while an angle formed by the direction of ultrasonic image formation in the second reception processing as shown in FIG. 7(a) is −θ relative to the direction of a perpendicular line of the surface on which the ultrasonic transducers 10a are arranged. As described above, by synthesizing the reception signals obtained based on the different directions of ultrasonic image formation, the effect of reducing speckle patterns can be obtained in one real transmission similarly in the case of the spatial compounding method.

The scan control unit 11 controls the reception beamformer 22 such that ultrasonic images are synthesized based on plural sets of reception signals with respect to the common point P, which signals are outputted from the reception signal processing units 21 and derived from the ultrasonic transducers in the plural reception apertures receiving ultrasonic echoes. The reception beamformer 22 synthesizes the plural sets of reception signals with respect to the common point P, which signals are outputted from the reception signal processing units 21 and derived from the ultrasonic transducers in the plural reception apertures receiving the ultrasonic echoes, and performs reception focusing processing on the synthesized reception signals to generate a sound ray signal representing a sound ray passing through the point P.

The synthesis of ultrasonic images can be also realized by synthesizing sound ray signals or synthesizing image signals, however, it is most desirable to directly synthesize reception signals obtained by receiving ultrasonic echoes for simplification of calculation. That is, the reception beamformer 22 as shown in FIG. 1 performs reception focusing processing by providing respective delays to the reception signals on the reception curves as shown in FIG. 4(b) and the reception signals on the reception curves as shown in FIG. 7(b), and adding those reception signals to one another. By the reception focusing processing, sound ray signals in which the focus of the ultrasonic echoes is narrowed are formed. The image signal generating unit 23 generates a B-mode image signal based on the sound ray signals.

1. An ultrasonic diagnostic apparatus comprising:
an ultrasonic probe including plural ultrasonic transducers for transmitting ultrasonic waves toward an object to be inspected according to drive signals and receiving ultrasonic echoes propagating from the object to output reception signals;
a drive signal generating unit for supplying the drive signals to said plural ultrasonic transducers, respectively;
a reception signal processing unit for processing the reception signals respectively outputted from said plural ultrasonic transducers;
a reception beamformer for performing reception focusing processing on the reception signals outputted from said reception signal processing unit to generate sound ray signals;
image signal generating means for generating an image signal based on the sound ray signals generated by said reception beamformer; and
scan control means for controlling said drive signal generating unit such that an ultrasonic beam with an intensity distribution having a center in a first direction is transmitted from a selected first group of ultrasonic transducers, and controlling said reception beamformer such that an image signal representing an ultrasonic image in a second direction different from said first direction is generated based on reception signals outputted from said reception signal processing unit and derived from a selected second group of ultrasonic transducers which receive ultrasonic echoes.

2. The ultrasonic diagnostic apparatus according to claim 1, wherein said scan control means controls said reception beamformer to generate a sound ray signal representing a sound ray passing through a point by phase-matching and adding reception signals outputted from said reception signal processing unit and derived from the second group of ultrasonic transducers which receive ultrasonic echoes generated due to ultrasonic waves transmitted from the first group of ultrasonic transducers, based on a relationship between directions of the ultrasonic waves transmitted from the first group of ultrasonic transducers to the point and reception directions of the second group of ultrasonic transducers which receive ultrasonic echoes generated from the point due to the ultrasonic waves.

3. The ultrasonic diagnostic apparatus according to claim 1, wherein said scan control means controls said drive signal generating unit such that ultrasonic beams are transmitted at plural times from ultrasonic transducers in sequentially selected plural transmission apertures, and controls said reception beamformer such that an image signal representing an ultrasonic image in the second direction different from the first direction is generated based on plural sets of reception signals outputted from said reception signal processing unit and derived from ultrasonic transducers in a reception aperture which receive ultrasonic echoes generated by the ultrasonic beams transmitted at plural times.

4. The ultrasonic diagnostic apparatus according to claim 1, wherein said scan control means controls said reception beamformer such that ultrasonic images are synthesized based on plural sets of reception signals with respect to a common point, said plural sets of reception signals being outputted from said reception signal processing unit and derived from ultrasonic transducers in plural reception apertures which receive ultrasonic echoes.

5. The ultrasonic diagnostic apparatus according to claim 4, wherein said reception beamformer performs reception focusing processing on the plural sets of reception signals with respect to the common point to generate the sound ray signals.

6. The ultrasonic diagnostic apparatus according to claim 1, wherein said scan control means controls said reception beamformer such that an image signal representing an ultrasonic image in a space region, in which energy of the ultrasonic beam transmitted from the first group of ultrasonic transducers not less than one tenth of the energy in the first direction exists, is generated.

7. The ultrasonic diagnostic apparatus according to claim 1, wherein:
said reception beamformer performs plural kinds of reception focusing processing on the reception signals outputted from said reception signal processing unit to generate plural kinds of sound ray signals;
said image signal generating means generates plural image signals based on the plural kinds of sound ray signals generated by said reception beamformer; and
said ultrasonic diagnostic apparatus further comprises a display unit for displaying plural ultrasonic images represented by the plural image signals generated by said image signal generating means.
